(19) United States
(12) Patent Application Publication
(10) Pub. No.: US 2014/0371574 A1
(43) Pub. Date: Dec. 18, 2014

(54) NONCONTACT ELECTROPHYSIOLOGICAL MEASUREMENT AND IMAGING OF THE HEART

(71) Applicant: University of Pittsburgh - of the Commonwealth System of Higher Education, Pittsburgh, PA (US)

(72) Inventors: Vladimir Shusterman, Pittsburgh, PA (US); Barry London, Iowa City, IA (US); Erik Branin Schelbert, Allison Park, PA (US)

(21) Appl. No.: 14/345,204

(22) PCT Filed: Sep. 17, 2012
(86) PCT No.: PCT/US12/55741

§ 371 Date: Mar. 14, 2014

Related U.S. Application Data
(60) Provisional application No. 61/535,584, filed on Sep. 16, 2011.

(51) Int. Cl. A61B 5/00; A61B 8/02; A61B 5/0402; A61B 5/02; A61B 5/021

(52) U.S. Cl. CPC A61B 5/7285; A61B 5/02028; A61B 5/021; A61B 5/0402; A61B 8/02
USPC 600/411; 600/410; 600/413

(57) ABSTRACT

Method and system for noncontact electrophysiologic imaging of the heart. The methods may employ the magnetization and its relaxation-based measurements, sensitive or specifically sensitized to the properties of cardiac electrical activity, to determine the spatio-temporal distribution of cardiac electromagnetic field and cardiac electrical potentials, and to display such spatio-temporal distribution (image) for assisting in the identification of the regions with abnormal cardiac electrical activity. In one embodiment, the system uses external magnets, gradient magnetic fields and radio-frequency waves, such as those commonly used for MRI, to generate the magnetic resonance. The system synchronizes scanning to the cardiac cycle using a measure of cardiac activity (e.g., electrocardiogram, ultrasound, ballistocardiogram, arterial pressure or cardiac sounds) and examines the difference between the cardiac electromagnetic properties (magnetization and relaxation) modulated by the oscillating radio-frequency fields and/or gradient fields of different orientations and magnitudes in the presence and absence of the cardiac electrical currents.

### CROSS-REFERENCE TO RELATED APPLICATION

This application claims the benefit of the earlier filing date of U.S. Provisional Application Ser. No. 61/535,584 filed on Sep. 16, 2011.

### GOVERNMENT FUNDING

This invention was made with government support under grant #OD003819 awarded by the National Institutes of Health. The government has certain rights in the invention.

### BACKGROUND OF THE INVENTION

1. Field of the Invention

This invention relates to the field of medical imaging and diagnosis, and more specifically to a method and system for noncontact imaging of the electrophysiological activity of the heart.

2. Description of the Background

Sudden cardiac death is a major public health problem and the primary cause of death in the industrialized world, claiming over 300,000 lives every year in the United States. It is usually caused by ventricular tachyarrhythmias, an abnormal heart rhythm that originates from the ventricles (the lower chambers of the heart). Another common arrhythmia that originates from the atria (the upper chambers of the heart) and can lead to major complications, including stroke, is atrial fibrillation. The “gold-standard” diagnostic modality in cardiac electrophysiology is cardiac electrophysiologic study. This is an invasive and highly complex procedure, which can be performed only in specialized hospitals by physicians trained in cardiac electrophysiology. This procedure requires advancing the wires (catheters) through the blood vessels into the cardiac cavity and/or cardiac blood vessels for measuring electrical activity from different regions of the heart. This procedure has associated risks of complications, requires significant time and exposure to ionizing radiation (for imaging the wire positions in the heart). Furthermore, the access to the different regions of the heart is limited to the largest cardiac vessels; advancing the catheters into the left part of the heart is associated with additional technical difficulties and risks of medical complications. The goal of the procedure is to localize the regions with abnormal electrical properties and correct these abnormalities (e.g., using some form of physical energy, referred to as the ablation procedure).

To obviate the shortcomings and technical difficulties associated with this invasive procedure, an alternative, noninvasive cardiac electrophysiologic (or electrocardiographic) imaging has been developed using a combination of the electrocardiographic (ECG) measurements obtained from the body surface and additional geometrical information about the location of the heart, which can be obtained using the computed tomography or magnetic resonance imaging of the heart (Pfeifer B, Hanser F, Seger M, Fischer G, Modre-Osprian R, Tilg B. “Patient-specific volume conductor modeling for non-invasive imaging of cardiac electrophysiology.” The Open Medical Informatics Journal, (2008) 2:32-41. Liu C, Skadsberg N D, Ahlberg S E, Swingen C M, Iaizzo P A, He B. “Estimation of global ventricular activation sequences by noninvasive 3-dimensional electrical imaging: validation studies in a swine model during pacing.” J. Cardiovasc. Electrophysiol. (2008) 19(5):535-540). Combining this information allows one to reconstruct the electrical potentials on the surface of the heart from those on the surface of the body, which has been shown to provide a reasonable reconstruction accuracy of 7-10 mm. Yet, this reconstruction problem is “ill-posed,” which means that it cannot be solved exactly; the solution is approximate and usually requires additional mathematical regularization, a priori knowledge and imposition of multiple constraints. An additional shortcoming is the necessity for a large number of ECG recording electrodes.

The magnetic resonance imaging (MRI) of the heart is a widely used imaging modality providing visualization of the cardiac anatomy and mechanical function. However, this imaging modality has never been used to obtain and visualize the spatio-temporal distribution of the electromagnetic fields generated by the heart.

Ehnholm in U.S. Pat. No. 5,250,900 (which is hereby incorporated by reference) teaches a method for nuclear magnetic resonance investigation of a repeated electromagnetic event using modulation of the nuclear spin polarization achieved during the recovery period between the final magnetic resonance signal detection period of one cycle and the initial signal generation radio-frequency (RF) pulse of the next cycle, which is performed by different exposures to RF radiation in the different periods.

Feasibility of tracking cardiac electrical activity in the heart using rotating-frame resonance in a low-power magnetic field has been demonstrated in small animals by Lindseth et al. (Lindseth B, Schwindt P, Kitching J, Fischer D, Shusterman V. “Non-contact measurement of cardiac electromagnetic field in mice using an ultra-small atomic magnetometer. Feasibility study.” Computers in Cardiology (2007):443-446, which is hereby incorporated by reference). Halpern-Manners et al. conducted phantom experiments in stronger fields (3-7 Tesla) and demonstrated the feasibility of high-resolution imaging of weak electrical currents (Halpern-Manners N W, Bajaj V S, Teisseyre T Z, Pines A. “Magnetic resonance imaging of oscillating electrical currents.” PNAS (2010) 107:8519-8524, which is hereby incorporated by reference). Truong et al. have shown that the electrical currents generated by the brain cells can be detected using the magnetic-field gradients synchronized with the currents of interest (Truong T K, Song A W. “Finding neuroelectric activity under magnetic-field oscillations (NAMO) with magnetic resonance imaging in vivo.” PNAS (2006) 103:12598-12601, which is hereby incorporated by reference).

Additional information regarding MRI experimental protocol design and implementation may be found in Handbook of MRI Pulse Sequences (edited by Matt A. Bernstein, Kevin F. King, and Xiaohong Joe Zhou (2004) Elsevier Inc.) and Magnetic Resonance Imaging: Physical Principles and Sequence Design (E. Mark Haacke, Robert W. Brown, Michael R. Thompson, & Ramesh Venkatesan (1999). Wiley, both of which are hereby incorporated by reference).

### SUMMARY OF THE INVENTION

The present invention provides systems and methods for noncontact electrophysiologic imaging of the heart that employs changes in electromagnetic properties (magnetization and relaxation) of cardiac tissues in the presence of electrical currents compared with those in the absence of electrical currents. The present invention may employ external and, preferably, gradient electromagnetic fields, as well as rotating or oscillating electromagnetic fields to obtain the spatio-temporal distribution of the electrical potentials/currents generated by the heart and its dynamics during the cardiac cycle.

The methods of the present invention may be used to assess cardiac electrophysiologic activity in a human or animal subject and may include the steps of obtaining images of cardiac tissue using MRI. The images may be synchronized to the cardiac cycle and consistently obtained from the same point in the cardiac cycle. A second image may be collected during a period of diastole when the heart is at rest. By subtracting the two images, a new “difference” image is obtained that reflects the electrophysiological activity of the heart. Through these techniques the present invention allows the assessment of atrial depolarization, atrial repolarization, ventricular depolarization, and ventricular repolarization.

To obtain images of cardiac electrophysiological activity, the cardiac tissue may be sensitized to the presence of electrical currents using spin-lock pulse sequences, gradient-switching pulse sequences, rotating-frame resonance magnetizations, or other imaging protocols that are well known to those of skill in the art. The magnetization may be accomplished through an external magnet, such as the magnets used traditionally in MRI. In other embodiments, the present invention may be implemented using the Earth's magnetic field, atomic magnetometers and highly sensitive magnetic microsensors.

The cardiac cycle of the subject may be measured using electrocardiogram, ultrasound, cardiac sounds, arterial pressure, ballistocardiogram, and other methods well known in the art. In certain embodiments, the imaging of the heart is gated by information about the cardiac cycle such that the imaging pulse sequence and subsequent measurement are synchronized to the cardiac cycle.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 provides a schematic of an electrocardiogram with examples of initiation times for MRI sequence origination;

FIG. 2 displays a diagram of a spin-lock pulse-sequence that may be applied to a subject at various times during a cardiac cycle;

FIG. 3 shows the spin-lock pulse-sequence diagram of FIG. 2 with the ordinate axis amplified; and

FIG. 4 displays a diagram of a gradient-switching (diffusion-weighted) pulse sequence that may be applied to a subject at various times during a cardiac cycle.

### DETAILED DESCRIPTION OF THE INVENTION

It is to be understood that the figures and descriptions of the present invention have been simplified to illustrate elements that are relevant for a clear understanding of the invention, while eliminating for purposes of clarity, other elements that may be well known.

The present invention provides a system and method for noncontact electrophysiologic (electrocardiographic) imaging of the heart using the changes in magnetization and its dynamical properties (relaxation) of the cardiac tissues in the presence of electrical currents compared with those in the absence of electrical currents. The present invention may employ external and, preferably, gradient electromagnetic fields to obtain the spatia-temporal distribution of the electrical potentials/currents generated by the heart and its dynamics during the cardiac cycle. The external magnetic fields can be generated by external magnets, such as those typically used in magnetic resonance imaging (MRI). In addition, the electromagnetic fields can be also generated by the radio-frequency (RF) transmitting coils (solenoids). These RF-generated, rotating or oscillating electromagnetic fields (B₁) are usually applied orthogonally to the main external magnetic field (B₀) to generate transverse magnetization (Mₜ). The Mₜ dynamics (relaxation) of the cardiac tissues, as measured by the receiving RF-coil (antenna), are used to generate the MR-image of the heart. Similarly, this invention can use an atomic magnetometer, which utilizes Larmor precession of the atoms driven by an electromagnetic field oscillating at the Larmor frequency in the presence of a static magnetic field, applied orthogonally or at some angle to the oscillating field. The magnetometer can measure small changes in the static field produced by the cardiac electrical activity.

While generally described here within the context of MRI image collection, the present invention may also be implemented using the Earth's magnetic field. The Earth's magnetic field may be used, essentially, for replacing the MR-magnet utilized in MRI image collection. The strength of the Earth's magnetic field is, of course, much lower than the strength of the MR-magnet, and accordingly low-field or micromagnetic sensors may be used to collect data from the subject.

As stated above, the method and system of the present invention involves an application of the oscillating (rotating) electromagnetic field Bₜ, also referred to as the radio-frequency (RF)-field. In one embodiment of the present invention, the low-power RF is applied for a relatively long time (>5 ms) to generate a relatively low-magnitude magnetic field B₁. This method is referred to as the spin-lock or the rotating-frame resonant mechanism. The power of B₁ is selected via the Larmor equation (ω=γ*B₁, here ω is the Larmor frequency of the protons' precession, γ is the gyromagnetic ratio and B₁ is the applied magnetic field) to approximate the frequency of: 1) the cardiac cycle, 2) its subharmonics, 3) the frequency of the cardiac waveform (action-potential) upstroke. The RF-field of the spin-lock may be tailored (gaited) to the timing of the upstroke of the cardiac action potential or the corresponding waveforms of the surface electrocardiogram (e.g., P-wave for the atrium and R-wave for the ventricles).

An additional advantage of this rotating-frame resonant approach compared with the methods based on phase change (i.e., changes in the coherent rotation of transverse magnetization or, equivalently, the phase coherence of the rotating (precessing) protons contributing to the transverse magnetization) is its independence from the directional and spatial variability and cancellation effects that impede phase-based imaging. An additional sensitization of the image can be achieved by applying repetitive gradient switching, synchronized with the electrical potentials/currents of interest.

While not wishing to be tied to theory, the information below provides a context helpful for understanding the present invention. The physical principles form the basis for estimates of the total and transverse magnetization (determining the MR-image intensity) of cardiac tissue within the context of the present invention. The principles relate to the externally applied electromagnetic fields and internal electromagnetic field generated by the heart. Internal electrical currents generated by the heart cause dephasing (loss of coherence) of the rotating magnetization (and precessing protons) in the transverse plane (relative to the direction of the B₀ magnetic field). Since the total magnetic moment μinside an MRI voxel in the transverse plane is the integral of the transverse magnetization over the volume of the voxel (Heller L, Barrowes B E, George J S. “Modeling direct effects of neural current on MRI.” Human Brain Mapping (2009) 30:1-12), the internal, bioelectric current changes both the magnitude and phase of μ, and consequently that of the MR signal. Thus, the magnetic field at any position can be written as the sum of the external field, B₀\hat{z}, and B′, the field generated by a bioelectric current. In high-field MR scanners, B′<<B₀, Without the relaxation term (which is relatively small), the Bloch-Torrey equation gives a simple estimate of the total magnetization (Torrey H C. “Bloch equations with diffusion terms.” Phys Rev (1956) 104:563-565):

$$\frac{\partial M}{\partial t} = γ M \times B + D {∇}^{2} M \qquad ( 1 )$$

Without the diffusion term, M precesses about the direction of the total field B with angular frequency γ|\dot{M}×B|. Calling M₀ the magnetization due to external field, and assuming that it is initially in the x-y plane, it evolves with time according to:

M₀₊(t)=M₀ₓ(t)+iM _{0y}(t)=M₀₊(t=0)e^{-iγB}^{₀}^{t} (2)

Neglecting the second order transverse component of B′, the evolution of magnetization can be approximated by M₊(r, t)=M₀₊(t)e^{-iΦ(r,i)}, where the additional phase due to bioelectrical activity is:

Φ(r,t)=γ∫₀′B′_{z}(r,t′)dt′,

where γ is the gyromagnetic moment of a proton −(2.67×10⁸)/Ts. Hence, a phase shift at biologically relevant magnetic field strength (˜nT to microT) would produce very small phase shifts. The MR signal is proportional to the net transverse magnetic moment in the volume V of a voxel:
μ₊(t)=∫_{γ}d³rM₊(r, t). Therefore, the ratio of this magnetic moment in the presence/absence of the bioelectric field is:

$${μ}_{+} ( t ) / {μ}_{0 +} ( t ) = \frac{1}{V} {\int }_{V} {d}^{3} r {e}^{- i Φ ( r , t )} \qquad ( 3 )$$

For the magnetic field calculation, the present invention employs the Biot-Savart law (in an adapted and simplified form, as shown in Blagoev K B, Mihaila B, Travis B J, Alexandrov L B, Bishop A R, Ranken D, Posse S, Gasparovic C, Mayer A, Aine C J, Ulbert I, Morita M, Muller W, Connor J, Halgren E. “Modelling the magnetic signature of neuronal tissue.” Neuroimage (2007) 37(1):137-48):

$$B ( {x}_{m} ) = \frac{{μ}_{0}}{4 π} {\sum }_{j = 1}^{N} \frac{{i}_{j} {r}_{mj} \times {dz}_{j}}{{r}_{mj}^{3}} \qquad ( 4 )$$

where N is the number of biological cells, rₘⱼ is the distance vector from monitoring point m to center of cell j, the magnitude of rₘⱼ is rₘⱼ, dzⱼ is the j-th cell line element vector, iⱼ is the instantaneous (constant over the length of a cell) current in cell j, μ₀ is the magnetic permeability in space and B(xₘ) is the magnetic field at point xₘ outside the cells. Alternatively, the magnetic field, B, may be estimated by using the Maxwell-Faraday equation:

$${∮}_{\partial S} E \cdot d 1 = - \frac{\partial {Φ}_{S} ( B )}{\partial t} ,$$

a line integral of the electric field, E, along the boundary ∂S of a surface, S (i is a vector element of the boundary curve).

As shown by Truong et al., assuming, as a first approximation, that the deformation is elastic (i.e., follows Hooke's law—displacement is proportional to the applied force and inversely propotional to Young's modulus of the elastic material), the ratio of the signal intensity with and without Lorentz force in a voxel of dimensions L×M will be computed using the following practical estimate:

$$R = \frac{\sqrt{\begin{matrix}{[ {\int }_{o}^{M} {\int }_{Δ {l}_{ma x} ( M - z ) / M}^{L} {ρ}^{′} ( z ) cos φ ( x , z ) d x d z ]}^{2} + \\ {[ {\int }_{o}^{M} {\int }_{Δ {l}_{ma x} ( M - z ) / M}^{L} {ρ}^{′} ( z ) cos φ ( x , z ) d x d z ]}^{2}\end{matrix}}}{{\int }_{o}^{M} {\int }_{Λ {l}_{ma x} ( M - z ) / M}^{L} ρ d x d z} \qquad ( 5 )$$

where ρ is the spin density in the absence of electrical currents, φ is a phase shift due to applying a sequence of oscillating gradients (synchronized with the current, which in the context of the present invention is equivalent to being synchronized to the cardiac cycle) and ΔIₘₐₓ is the maximum phase displacement in the presence of an electrical current.

The fundamental difference between the study of brain currents by Truong et al. and the study of cardiac currents relates to the complex movements and deformations of the heart. In addition, cardiac muscle is not purely elastic. Therefore, the forces are preferably examined using at least 2 and possibly >2 different magnetic field gradients to estimate the differences between the displacements produced by the application of these gradients from different directions. Obviously, the displacements caused by the cardiac contractions will be the same for all different gradients. Therefore, the differences in the displacements in response to different magnetic-field gradients will identify the Lorentz forces in the current-carrying areas of the heart. This, in turn, will allow the computation of the spatiotemporal distributions of the cardiac electrical potentials of the beating heart. Since the heart muscle is not purely elastic, the distribution of the forces may be corrected by using the functional F(x, z) representing cardiac contraction and movement. Finally, the effect of the Lorentz force may be estimated using the differences between the products R*F(x,z) for the gradients applied in different directions and the centroid product R̅*̅F̅(̅x̅,̅ z̅)̅ obtained by calculating the Euclidean distance (vector magnitude) or Mahalanobis distance of the products R*F(x, z) obtained by applying the magnetic field gradients in different directions. This practically important estimate may be made using the following quantity:

$$\sqrt{\frac{1}{N - 1} {{\sum }_{1}}^{N} {( R * F ( x , z ) - \overline{R * F ( x , z )} )}^{2}} . \qquad ( 6 )$$

It is also possible to calculate the spatiotemporal dynamics of the cardiac magnetic and electrical fields using other Maxwell equations (which can be used in combination with the Lorentz equation as well). Since these equations are well described, they are provided here in a single brief form (a detailed description involves the constitutive equations for each cardiac tissue and cardiac cavity). Specifically, because the magnetic field H is known (or can be measured with high precision by an MR-compatible magnetometer), one can determine the magnetic vector potential A:

μH=V×A

Therefore, the total displacement current Jₜₒₜ is also determined using a straightforward calculation:

∇×H=Jₜₒₜ

If the electrical conductivity σ is known (approximately), one can also estimate the electromotive force (i.e., electrical field E):

$$E = \frac{1}{σ} J$$

Finally, since the instantaneous particle velocity is known, the electrical potentials φ is calculated using the following equation:

$$E = μ v \times H - \frac{\partial A}{\partial t} - ∇ φ \qquad ( 7 )$$

Multiple confounding factors (e.g., electromagnetic interference and background thermal noise, mechanical movements, tissue heterogeneities, moving blood, among others) may affect the accuracy and stability of these computations. Additional mathematical tools may be used to sharpen the accuracy of the calculations by taking into account the confounding factors.

The method of the present invention may employ the following contrast mechanisms. Each mechanism can be used separately or in combination with other mechanisms. In addition, preparatory modules (e.g., the inversion-recovery, magnetization transfer, chemical shift, spatial saturation and combinations of those modules) can be applied within each mechanism's pulse sequence. The readout can be accomplished using either gradient-echo, spin-echo, or free precession applied in two or three dimensions. In addition, the contrast mechanisms described herein can be combined with diffusion-sensitized (weighted) imaging and/or diffusion-tensor imaging to obtain the spatial information about the location of specific anatomical structures, for example, the cardiac electrical-conduction system. To shorten data acquisition time, the sequences can be implemented using fast and parallel imaging approaches (e.g., turbo spin echo). The approaches described below can be also combined with the magnetic-resonance angiography (MRA) and flow compensation techniques, as well as phase-contrast MRA and contrast-enhanced MRA, to obtain information on the blood flow in the heart and/or blood vessels. Other contrast-enhanced imaging modalities well known to those of skill in the art may be also used in conjunction with methods described below.

Contrast mechanism I. Rotating-frame resonance and spin-locking for the imaging of cardiac electrical activity.

In this approach, which is also known as the “T₁-rho relaxation”, the power (and, therefore, Larmor frequency) of the rotating, orthogonal magnetic field is matched (spin-locked) to the frequency of the oscillating electrical currents generated in the heart. This results in a rotating-frame resonance, in which the protons associated with electrical currents of interest, experience additional loss of the transverse magnetization and loss of signal intensity in the corresponding part of the MR image where electrical current is flowing.

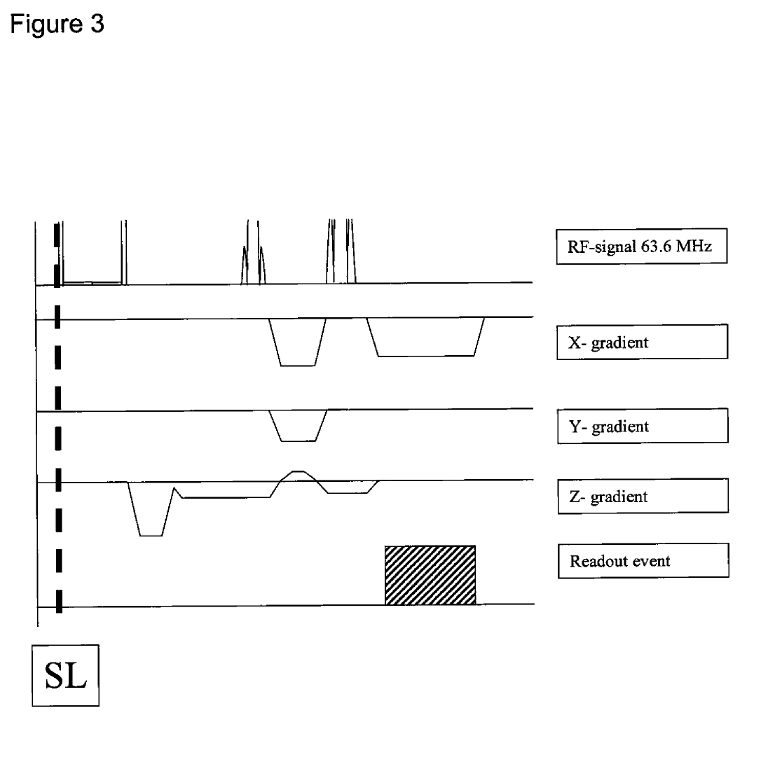

As stated above, the low-power RF may be applied for a relatively long time (>5 ms) to generate a relatively low-magnitude magnetic field B₁, as exemplified in FIG. 3. This method is referred to as the spin-lock or the rotating-frame resonant mechanism. The power of B₁ is selected via the Larmor equation (ω=γ*B₁, where ω is the Larmor frequency of the protons' precession, γ is the gyromagnetic ratio and B₁ is the applied magnetic field) to match the frequency of: 1) the cardiac cycle, 2) its subharmonics, 3) the frequency of the cardiac waveform (action-potential) upstroke. The RF-field of the spin-lock is tailored (gaited) to the timing of the upstroke of the cardiac action potential or the corresponding waveforms of the surface electrocardiogram (e.g., P-wave for the atrium and R-wave for the ventricles). The images generated as described above are compared with the reference images generated during the diastole of the cardiac cycle, when no electrical activity is present. The difference images show the net effect and location of the cardiac electrical activity.

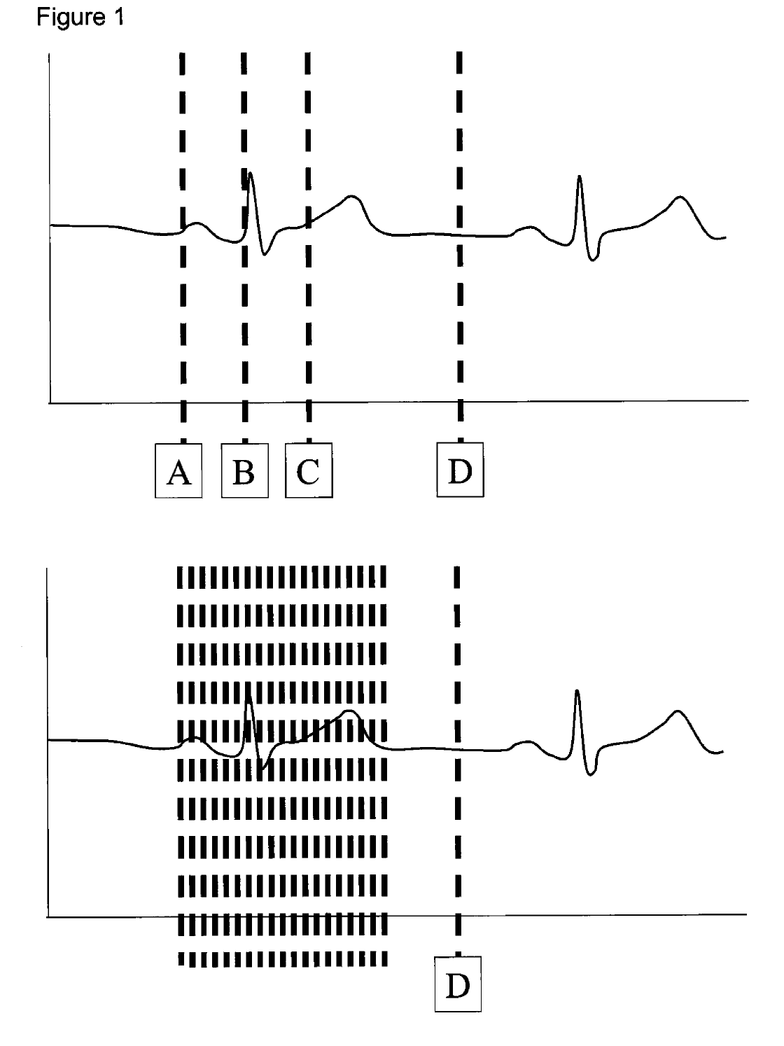

FIG. 1 provides examples of the initiation times for the spin-lock and/or gradient switching (described below) sequences as referenced to the contractions of the hart shown in a typical electrocardiogram. Line A shows the initiation time for a sequence that can be applied for imaging of the atrial electrical activity. Line B shows the initiation time for a sequence that can be applied for the imaging of the ventricular electrical activation. Line C shows a possible initiation time for the sequence that can be applied for imaging of the cardiac ventricular electrical repolarization phase (i.e., recovery). Line D shows a possible initiation time for a sequence that can be used as a reference for the sequences collected at any of times A, B, or C. The sequence initiated at time D may occur at any point during the diastole (the quiescent period of the cardiac cycle).

The MR-pulse sequences may be triggered (gaited) using an electrocardiogram to ensure that the MR pulse sequence starts at the same time within the cardiac cycle (FIG. 1). For purposes of gating the MR pulse sequence, the cycle of the heart may be assessed through any one of many commonly employed methods, including electrocardiogram, ultrasound, cardiac sounds, arterial pressure, and ballistocardiogram. The precise start time of the pulse sequence depends on the specific region of interest within the heart. For example, to study the electrical activity in the atria, the MR-sequence (more precisely, the spin-locking part of the sequence or the gradient-switching part) can be triggered by the onset of the electrocardiographic P-wave (as shown by line A in FIG. 1). To study ventricular depolarization, the MR-sequence's spin-lock or gradient-switching can be triggered by the electrocardiographic R-wave (as shown by line B in FIG. 1). To study ventricular repolarization (recovery), the MR-sequence can be triggered by the ST-segment or the beginning of the electrocardiographic T-wave (as shown by line C in FIG. 1). To obtain a reference MR-image, the MR-sequence can be gated by the electrocardiographic diastolic (TP) interval, as shown by line Din FIG. 1. The resulting image is obtained by subtracting the reference MR-image (i.e., image collected at Line D) from the respective “active” image obtained during the electrically active period of interest within of the cardiac cycle (e.g., line A, B or C in FIG. 1).

The bottom panel of FIG. 1 shows that the sequence initiation times (dashed lines) can be shifted consecutively in small time intervals (1 to 50 ms) in a series of scans to span the entire time cycle of the cardiac electrical activity. After the difference images are obtained at different times within the cardiac cycle, these images may be combined to obtain the spatio-temporal distribution (map, spread, dynamics) of cardiac electrical activity during the cardiac cycle. The origins and pathways for cardiac arrhythmias and other diseases of the heart will be analyzed using these maps. For example, abnormal cardiac conduction (depolarization) will be manifested by the regions of slow conduction, irregular waves of electrical excitation, the presence of abnormal patterns of electrical excitation (e.g., rotating waves or small wavelets in the case of atrial fibrillation) or abnormal (accessory) pathways/spread of electrical activation (e.g., Wolf-Parkinson-White syndrome). Similarly, a myocardial scar caused by myocardial infarction or fibrosis can block or decrease the speed of propagating electrical activity and distort normal patterns of electrical excitation. The spread of electrical activity between different segments (walls) of the ventricles or between the left and right ventricles of the heart may lose normal, synchronous pattern and become dyssynchronous in patients with heart failure (referred to as the cardiac electrical dyssynchrony). Other examples of abnormal electrical activity include, but are not limited to, spiral and reentrant waves of electrical activity, clusters of cells generating ectopic electrical activity (extrasystoles), abnormal patterns of electrical repolarization (recovery), such as the long QT-syndrome, Brugada syndrome and changes in the amplitude of the electrocardiographic ST-segment, which can be caused by myocardial ischemia, abnormal electrolyte levels and other abnormalities.

The abnormal pattern of electrical activity (more precisely, electrical activation) of the cardiac tissues in those morbid conditions will impact the magnetic properties of the respective tissue segments in which the electrical activation is present during the imaging sequence. This will in turn be reflected in differences between the images collected during the MR paradigm from subjects with abnormal patterns of electrical activity (electrical depolarization or repolarization) compared with subjects with normal patterns of electrical activity.

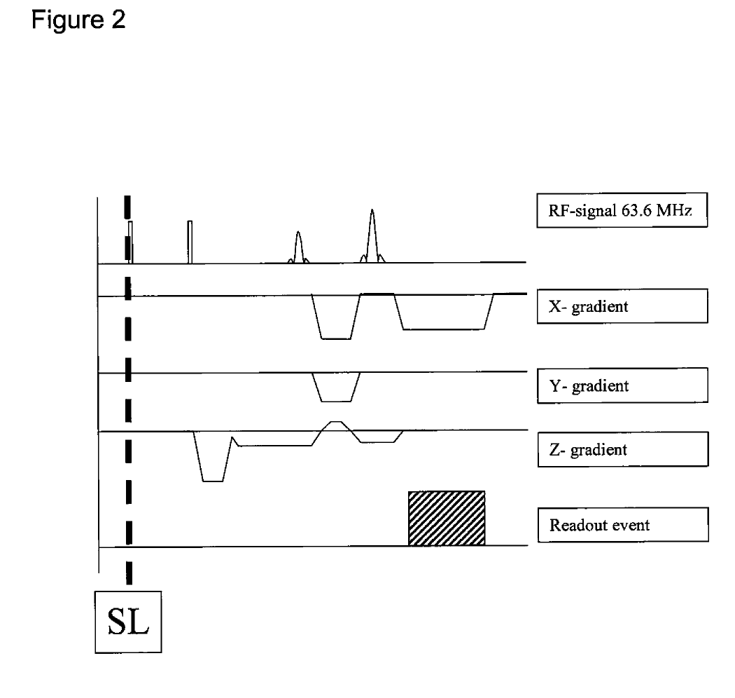

To collect MR images for the assessment of the cardiac electrophysiological activity, several different types of pulse sequences may be used to sensitize the images to the cardiac electrical currents, including spin-lock sequences and synchronized gradient switching sequences. FIG. 2 provides an example of a spin-lock (T1-rho) pulse-sequence diagram, which may be applied at any of the various times approximated by lines A, B, C, and D in FIG. 1. The line labeled SL indicates the beginning of the spin-lock module, which may occur at any of the times marked by lines A, B, C, and D in FIG. 1. The panels show (from top to bottom), the RF-signal, X-gradient, Y-gradient, Z-gradient, and readout events that may occur during tissue stimulation and data collection.

FIG. 3 shows the spin-lock (T1-rho) pulse-sequence diagram shown in FIG. 2 with the ordinate axis of the RF-signal amplified to show a low-amplitude signal during the period of spin lock. This low-amplitude RF signal creates magnetic field B₁ as discussed above.

Although the spin-locking pulse sequences have been previously used for the imaging of electrical activity in the brain, such sequences have not been applied for the imaging of electrical activity in the heart. There are important differences between the properties of electrical activity in the brain and in the heart, which lead to the differences in the imaging approaches: 1) the electrical activity of the brain is continuous (which forms the basis for the traditional spin-lock approach), whereas the cardiac electrical activity has a period of macroscopic electrophysiological quiescence (during the diastole); and 2) the electrical activity of the heart is significantly slower than that of the brain, which makes it very difficult to acquire several cycles of electrical activity (also a principal requirement of the traditional spin-lock) during a single cardiac cycle. Therefore, the classical spin-lock approach, which has been previously used for the brain imaging, cannot be simply extrapolated to the imaging of the cardiac electrical activity.

Contrast Mechanism II. Synchronized Gradient Switching (SGS) or Diffusion-Weighted Imaging of Cardiac Electrical Activity

The heart can be viewed as an electrical conductor, with an electrical current propagating from the sinus node to the atrio-ventricular node and to the ventricles. In a magnetic field, the heart will experience additional dephasing of the protons in the areas of the propagating electrical currents. There are several mechanisms that can explain such dephasing. One mechanism of dephasing is the Lorentz force, which is equal to the vector (cross) product of the electrical current's vector and the strength of the magnetic field; it causes a small displacement of the current-carrying region in the direction of the cross-product. This, in turn, results in a displacement of the spins in this region and a loss of phase coherence, compared to those in the same region when the electrical current is not present (i.e., when the voltage gradient is equal to zero). Another mechanism of dephasing is related to the eddy currents, which are generated in the electrical conductors by the changes in magnetic fields, for example, switching of the gradient magnetic fields and/or changes in the magnetic fields generated by varying RF. The eddy currents are affected (increased or decreased, depending on the direction of the current) by the internal electrical fields/currents flowing in the conductor—particularly, the electrical activity of heart. Therefore, the amount of dephasing will be different for the conductors with and without flowing currents. The principles and rationale for using the SGS-sequence are similar to those for the diffusion-weighted imaging (with a notable difference that diffusion-weighted imaging sequences have not been used for the imaging of electrical activity and, in particular, the cardiac electrical activity).

The direction of the dephasing generated by the mechanisms described above is different for different orientations of the magnetic field gradients. Therefore, applying such different orientations of the magnetic field gradients and analyzing the differences in the resulting dephasing reveals the areas carrying electrical currents at each time point within the cardiac cycle. From this information, it is straightforward to construct the spatiotemporal distribution of the cardiac electrical potentials generated by the heart.

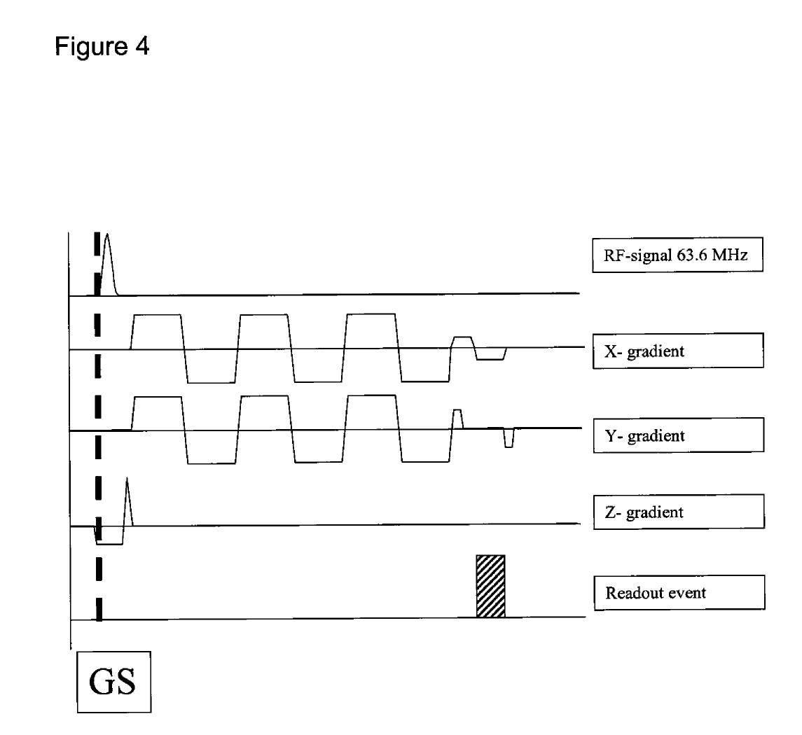

The RF-pulse sequence is preferably synchronized with the timing of the cardiac cycles, and the resulting signals obtained during several cardiac cycles can be averaged to achieve an improved signal-to-noise ratio. Furthermore, the signal can be amplified by using a sequence of oscillating pulses as shown in FIG. 4. In FIG. 4, the line GS marks the time of the RF-pulse at the beginning of a gradient-switching pulse sequence. That sequence may be applied at various times throughout the cardiac cycle and may occur at any of the times marked by lines A, B, C, and D in FIG. 1. The panels show (from top to bottom), the RF-signal, X-gradient, Y-gradient, Z-gradient, and readout events that may occur during tissue stimulation and data collection.

### EXAMPLE 1

Preliminary experiments were performed in vitro using a phantom heart. The experiments were designed to assess whether physiologically relevant currents produced magnetic fields that are reliably measurable using the techniques of the present invention. The theory and feasibility of the present invention were confirmed by the present experiments.

Experiments were performed using a box-shaped phantom (approximate dimensions: 20×15×10 cm) fabricated from 2.4% agar and 0.5 mmol of copper sulfate. The phantom possessed MR characteristics matching those of a cardiac tissue (T1=877-950 ms with regional heterogeneity; T2=70 msec). A thin 99.5%-carbon thread (approximate dimensions: 75×200 microns) with the resistance matching that of a cardiac tissue (˜0.3-1 kOhm) was run through the phantom and connected to an arbitrary electrical function generator and a 100 MHz digital oscilloscope.

MR-images were acquired using a 1.5 Tesla MR scanner, using custom sequences described in the invention disclosure, for various amplitudes, frequencies and waveforms of the electrical signals (square pulses of alternating polarity, sine waves, triangular waves and other waveforms synchronized with the alternating MR gradients).

The location of the carbon thread was clearly visible on the difference images obtained by subtracting the baseline images (no electrical current) from the images obtained in the presence of electrical currents (signals). These results were reproducible for electrical signals having the magnitude (5 mV to 10 mV) and frequency (0.3 Hz to 3 Hz), consistent with those for cardiac electrical activity, as well as for the higher frequency (3 Hz to 1 kHz) and amplitude (10 mV to 10 V) signals. The signal-to-noise ratio and visibility of the current-conducting area was improved by signal averaging (3 to 10 times). These results confirm that physiologically relevant currents flowing through an object having cardiac-like MR properties are easily discernable, thus confirming the implementation of the present invention.

Applications of the method and system of present invention include any and all types of electrophysiological testing and imaging for any and all types of electrophysiological abnormalities. An important but not limiting example of such applications is to measure the speed and/or path of spread of electrical activity through the heart in one, two, or three dimensions. This approach may be utilized for guiding cardiac resynchronization therapy and for prognosis and management of patients with heart failure. In particular, it may be utilized for the measurement of abnormal conduction in heart failure and/or cardiomyopathy.

Nothing in the above and attached descriptions is meant to limit the present invention to any specific materials, geometry, or orientation of elements. Many modifications are contemplated within the scope of the present invention and will be apparent to those skilled in the art. The embodiments disclosed herein were presented by way of example only and should not be used to limit the scope of the invention.

What is claimed is:

1. A method assessing cardiac electrophysiologic activity in a patient, comprising the steps of:
applying a first magnetic resonance pulse sequence to a heart of a subject at a point in a cardiac cycle where said heart is electrically active;
obtaining a first image of said heart at said point in said cardiac cycle where said heart is electrically active;
applying a second magnetic resonance pulse sequence to said heart of said patient at a point in said cardiac cycle where said heart is not electrically active, wherein said second magnetic resonance pulse sequence is the same as said first magnetic resonance pulse sequence;
obtaining a second image of said heart at said point in said cardiac cycle where said heart is not electrically active;
subtracting said second image from said first image to obtain a subtracted image of said heart; and
evaluating said subtracted image to ascertain said cardiac electrophysiological activity.

2. The method of claim 1, wherein said point in the cardiac cycle where said heart is electrically active is selected from the group consisting of atrial depolarization, atrial repolarization, ventricular depolarization, and ventricular repolarization.

3. The method of claim 1, wherein said first and second magnetic resonance pulse sequences are both a spin-lock pulse sequence.

4. The method of claim 3, wherein said spin-lock pulse sequence comprises a magnetic field matched to the frequency of oscillating electrical currents generated in the heart.

5. The method of claim 1, wherein said first and second magnetic resonance pulse sequences are both a gradient-switching pulse sequence.

6. The method of claim 5, wherein said gradient-switching pulse sequence is synchronized with said cardiac cycle.

7. The method of claim 1, wherein said first and second images of said heart are images of a region of said heart.

8. The method of claim 1, wherein said applying and obtaining steps are performed using at least one external magnet.

9. The method of claim 1, wherein said applying and obtaining steps are performed using at least one of the Earth's magnetic field, an atomic magnetometer, and a gradient magnetic field.

10. The method of claim 1, in which said applying and obtaining steps are performed using at least one gradient magnetic field with at least one temporal change in the field being consistently synchronized with at least one time point of the cardiac cycle.

11. The method of claim 1, wherein said patient needs electrophysiological evaluation of the heart due to at least one indication selected from the group consisting of an arrhythmia, history of arrhythmia, risk of arrhythmias, electrical dyssynchrony, abnormal conduction, depolarization abnormalities, repolarization abnormalities, suspected electrophysiological abnormalities of the heart, the measurement of abnormal conduction in heart failure, and the measurement of abnormal conduction in cardiomyopathy.

12. The method of claim 1, further comprising the step of measuring said cardiac cycle.

13. The method of claim 12, wherein said measuring step is achieved through electrocardiogram, ultrasound, cardiac sounds, arterial pressure, or ballistocardiogram.

14. The method of claim 12, further comprising the step of gating said applying and obtaining steps based on said measuring step.

15. The method of claim 14, wherein said gating synchronizes said applying and obtaining steps to said cardiac cycle.

16. The method of claim 1, wherein said subject is selected from a human subject and an animal subject.

17. The method of claim 1, wherein said heart is selected from the entire heart, atria of the heart, ventricles of the heart, a segment of the heart, a wall of the heart and tissue of the heart.

18. The method of claim 1, in which said applying and obtaining steps are performed using at least one rotating magnetic field with at least one temporal change in the field being consistently synchronized with at least one time point of the cardiac cycle.

19. A system adapted for cardiac electrophysiologic imaging of a heart of a patient comprising:
a collection unit for collecting information related to magnetic properties of a heart using at least one programmable sequence of MRI-based electromagnetic events selected from the rotating-frame resonance, spin-locking, and synchronized gradient switching;
an analysis unit for analyzing spatio-temporal dynamics of said information within a cardiac cycle to obtain the spatio-temporal distribution of at least one of an electromagnetic field and electrical potentials generated by the heart; and
a display for displaying said spatio-temporal distribution of electrical potentials in the heart to enable direct tracking of at least one electrophysiological process selected from a path and speed of cardiac electrical activation and detection of regions of abnormal cardiac electrical activity.
